(19) United States
(12) Patent Application Publication
(10) Pub. No.: US 2014/0187859 A1
(43) Pub. Date: Jul. 3, 2014

(54) ENDOLUMINAL INTRODUCER

(71) Applicant: Novadaq Technologies Inc., Mississauga (CA)

(72) Inventors: Christopher LEEUW, Vancouver (CA); John FENGLER, North Vancouver (CA); Danny Sherwinter, New York, NY (US)

(73) Assignee: Novadaq Technologies Inc., Mississauga (CA)

(21) Appl. No.: 14/140,370

(22) Filed: Dec. 24, 2013

Related U.S. Application Data
(60) Provisional application No. 61/745,682, filed on Dec. 24, 2012.

(51) Int. Cl. A61M 13/00; A61B 1/00; A61B 1/06; A61B 17/34

(52) U.S. Cl. CPC A61M 13/003; A61B 17/3472; A61B 1/00154; A61B 1/063; A61B 1/0638
USPC 600/114; 604/26

(57) ABSTRACT

An introducer for use during endoscopic procedures provides insufflation, washing, and aspiration functions, and provides for the protection of the endoluminal surface during laparoscopic examination of an anastomosis or suture line following low anterior resection of the bowel. The introducer may be designed for the insertion of an endoscope capable of white light and/or near infra-red fluorescence imaging into the rectum for analysis of an anastomosis following low anterior resection of the bowel.

### CROSS-REFERENCE TO RELATED APPLICATION

This application claims the benefit of priority of U.S. Provisional Application No. 61/745,682 filed Dec. 24, 2012, the disclosure of which is incorporated herein by reference.

### TECHNICAL FIELD

The present disclosure relates to an endoluminal introducer and, more particularly, to an introducer for the insertion of an endoscope capable of white light and/or near infra-red fluorescence imaging.

### BACKGROUND

Low anterior resection (LAR) is a common surgery of the rectum for rectal cancer. Such surgery is increasingly, although not exclusively, performed using minimally invasive surgical (MIS) techniques. With the advent of high-definition (HD) laparoscopy, intra-operative visualization during MIS has achieved new performance standards and enhanced the rectal surgeon's ability to assess and navigate the surgical field. Such endoscopes are typically used in conjunction with high definition camera systems to produce real time video images which can be displayed on an appropriate high definition video monitor and recorded on an appropriate high definition recording medium (such as an HD video recorder).

Upon completion of an LAR surgery, the surgeon may want to follow up with an endoluminal examination of the suture line within the rectum. The purpose of the endoluminal examination is to confirm that the tissue surrounding the anastomosis is well perfused, as such perfusion indicates that the healing process will be successful and that leaks (i.e. leaks of fecal matter into the peritoneum and resulting complications) are less likely to occur. Failure of the anastomosis, often taking the form of anastomotic dehiscence, is a devastating complication of LAR surgery. Sepsis resulting from fecal leaks into the peritoneum can result in acute effects, and even death, in the short term. Long term morbidities associated with anastomotic failure include stricture formation, bowel dysfunction and an increased chance of cancer recurrence. Ischemia of the tissue surrounding the anastomosis is the likely cause of the majority of complications.

Post-resection endoluminal examinations are typically performed with a rigid sigmoidoscope—a much lower resolution optical imaging device than the HD laparoscope used during the surgical procedure. To optimally view the suture line or anastomosis in such follow-up endoluminal examinations, it would be preferable to utilize the HD laparoscope to obtain the highest quality image. Unfortunately, laparoscopes do not have a number of practical features that are required for such examinations. Most notably, insufflation of the rectum and the capability to wash and aspirate fluids from the endoluminal surface are required for proper imaging of the anastomosis and suture line.

Laparoscopes are rigid endoscopes and are typically composed of illumination optics and imaging optics contained in a stainless steel shaft that is 2 mm-15 mm in diameter. The illumination optics within the laparoscope are primarily composed of a fiber-optic light guide. The light guide leads light from an endoscopic illuminator to the distal tip of the laparoscope from which the light is emitted to illuminate the field of view.

The laparoscope imaging optics typically consist of a set of optical lenses that relay an image from the distal to the proximal end of the endoscope for viewing through an eyepiece or for imaging using an endoscopic camera. The optics at the distal tip of the laparoscope may be forward-looking with a zero degree (0°) angle of view, or side-looking (30° or 45° angle of view).

Alternative construction of laparoscopes includes versions in which a camera is built into the endoscope (either at the proximal or distal tip). Furthermore, some laparoscopes may contain a built in illumination source (i.e. a solid state source such as LEDs or laser diodes) to eliminate the need for a separate endoscopic illuminator. However, because laparoscopes are intended solely for intraperitoneal visualization during MIS and separate instruments are used for insufflation, washing and aspiration during such surgeries, laparoscopes are typically not equipped with the features (i.e. the working channels and controls) that provide such functions. Furthermore, laparoscopes have relatively sharp edges at the endoscope tip and may scrape the mucosal surface if introduced endoluminally without some additional device that protects the endoluminal surface from such abrasions.

There is a need, therefore, for a device that provides a laparoscope with the aforementioned features and capabilities for endoluminal examination. Specifically, it would be desirable to have an introducer for use with a laparoscope that has at least one of insufflation, washing and aspiration functions and that provides protection from the laparoscope for the endoluminal surface during use.

### SUMMARY

Described herein is an endoluminal introducer that can be used in conjunction with a laparoscope for endoluminal examination following LAR surgery. The introducer acts as a conduit for introducing the laparoscope into the rectum and enables the viewing of the endoluminal surface and surgical margin or anastomosis with the same HD endoscope that is used for intra-peritoneal viewing during surgery. The introducer contains all of the features required for examination of the endoluminal surface and may include channels for the washing and aspiration of liquids and for the introduction of insufflation air (or CO2 or other similarly appropriate insufflation gas) to expand the endoluminal space and completely examine the surgical anastomosis.

The introducer may be composed of a rigid medical plastic formed into a tube structure by molding, extrusion or other appropriate plastic manufacturing process. The introducer may contain multiple channels, with the laparoscope being inserted into the main channel and the other channels being utilized for insufflation, washing and aspiration of fluids from the endoluminal surface. Alternatively, the space between the laparoscope and the wall of the main channel may be used for such functions.

The introducer is approximately the length of a suitable laparoscope, such that when the laparoscope is inserted into the introducer, the tip of the endoscope may reach, but does not protrude from, the end of the introducer. The main channel (103) of the introducer may be sealed at the distal end with a transparent window. In most embodiments of the introducer, the tip of the laparoscope shall be sufficiently close to the end of the main channel of the introducer, so that the introducer does not enter the field of view seen through the laparoscope or block the illumination emitted by laparoscope.

In various embodiments, the proximal end of the introducer may contain a number of valves for controlling the insufflation, suction, and wash functions of one or more separate ancillary channels of the introducer. Insufflation air and wash water may be supplied by an air pump and water bottle built into the endoscopic illuminator or as standalone components. Alternatively plumbed-in air or CO2, water and vacuum lines in the operating room may be used.

In some embodiments, the main channel at distal end of the introducer does not necessarily have a window that seals and separates the laparoscope from the endoluminal space. In this arrangement, the laparoscope tip is exposed to the endoluminal surface and the introducer contains a circumferential seal between the exterior of the laparoscope and the interior surface of the main channel of the introducer so as to contain the insufflation air within the endoluminal cavity. Such an embodiment may also integrate the insufflation and main channels of the introducer into a single channel. Such an embodiment may also integrate a separate ancillary channel to direct a spray of wash water across the tip of the laparoscope or ancillary channels for the irrigation and aspiration of fluids.

The introducer of the present invention may be used with traditional, white-light endoscopes or with an endoscope capable of simultaneous white-light and near infra-red imaging. Use of the introducer with the latter type of endoscope provides for enhanced assessment of tissue perfusion in tissue adjacent to an anastomosis as compared to imaging using traditional endoscopy. In turn, this results in a reduction in the complications described above associated with anastomotic leakage.

Further advantages and embodiments are apparent from the appended drawings.

### BRIEF DESCRIPTION OF THE DRAWINGS

In the figures:

FIG. 1 shows an endoluminal introducer for use in conjunction with a laparoscope.

FIG. 2 displays several views of an endoluminal introducer with several channels that allow for multiple functionalities.

FIG. 3 shows an arrangement of channels and valves providing for insufflation, washing and aspiration according to an embodiment of the present invention.

FIG. 4 shows an arrangement of the introducer, a laparoscope and a seal allowing for appropriate insufflation during use of the present invention.

FIG. 5 displays a connection mechanism situated mid-way between the distal and proximal ends of the device allowing for assembly before insertion of the laparoscope.

FIG. 6 shows a seal feature of the connection mechanism of FIG. 5 allowing for maintenance of insufflation pressure during use of the device of the present invention.

FIG. 7 shows a removable handle for incorporation into the introducer of the present invention that may contain valves for the control of insufflation, washing and aspiration.

### DETAILED DESCRIPTION

With reference to the figures attached hereto and briefly referred to above, several preferred embodiments are now described in detail.

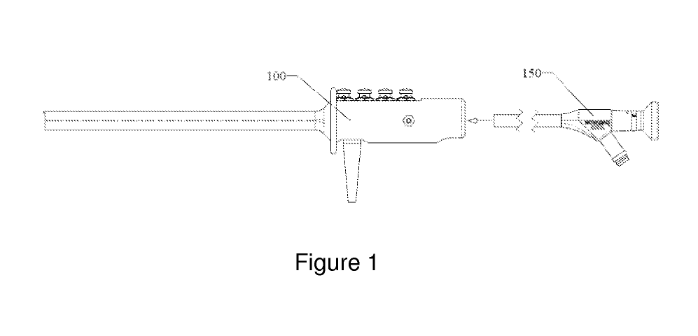

The invention described herein (FIG. 1) is an endoluminal introducer (100) that can be used in conjunction with a laparoscope (150) for endoluminal examination following LAR surgery. The introducer acts as a conduit for introducing the laparoscope into the rectum and enables the viewing of the endoluminal surface and surgical margin or anastomosis with the same HD endoscope that is used for intra-peritoneal viewing during surgery. The introducer incorporates all of the features required for examination of the endoluminal surface with a surgical endoscope. These features may include channels for the introduction of insufflation air (or CO2) to expand the endoluminal space and for the washing and aspiration of liquids so as to provide for the complete examination of the surgical anastomosis.

Furthermore, if the HD laparoscope is capable of near infrared illumination and imaging, the anastomosis may be viewed using an ICG imaging agent to highlight the perfusion of tissue at and around the area of the surgery. One such laparoscope is the Pinpoint® system (Novadaq Technologies Inc., Canada) that provides for simultaneous white-light and near infra-red illumination and imaging. This allows for enhanced visualization and assessment of the anastomosis and surgical margin over that which can be achieved with conventional white-light endoscopes.

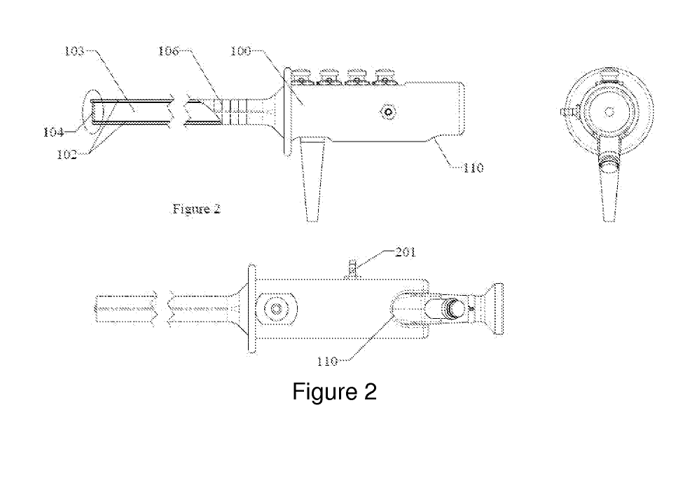

In one embodiment, (FIG. 2), the introducer (100) is composed of a rigid plastic formed into a tube structure by molding, extrusion or other appropriate plastic manufacturing process. The plastic may be selected from a medical plastic, polypropylene, polycarbonate, polyethylene, polystyrene, K-resin, or any other appropriate rigid plastic. The tube structure may be transparent or opaque. The introducer may contain a single main channel or a main channel with one or more ancillary channels (102)—the laparoscope being inserted into a main channel and the other channels being utilized for insufflation, washing and aspiration of fluids from the endoluminal surface. If the introducer has a single main channel that is open to the endoluminal space, then the space between the laparoscope and the tube wall may be utilized for insufflation, washing and aspiration of fluids from the endoluminal surface.

In many embodiments, the introducer is approximately the length of the laparoscope, such that when the laparoscope is inserted into the introducer, the tip of the endoscope reaches, but does not protrude from the end of the introducer. The main channel (103) of the introducer may be sealed at the distal end with a transparent window (104) and, if sealed, the main channel window may be transparent to UV, visible or near infra-red light. The tip of the laparoscope is sufficiently close to the end of the main channel of the introducer, so that the introducer does not enter the field of view seen through the laparoscope or block the illumination emitted by laparoscope.

The tip (105) of the introducer may be angled at 30°, 45°, or 90° to accommodate angle viewing laparoscopes. Ancillary channels for washing and aspiration are appropriately directed to terminate in the same direction as the viewing angle. A separate ancillary channel may be terminated to direct a spray of wash water across the window of the main channel.

The tip (105) of the introducer may be composed of a softer more compliant plastic than the remainder of the shaft of the introducer, (e.g. Teflon or a similar material) or may have rounded edges so as not to scrape the endoluminal surface when inserted.

The introducer may have markings (106) on the exterior surface to indicate the depth of insertion.

The proximal end of the introducer may have a feature (110) that seats the laparoscope light guide stem and maintains it in position such that the introducer and laparoscope will move together if rotated. This is especially useful in instances when side-viewing laparoscopes are used.

The proximal end of the introducer may have a connection point for the insufflation, aspiration and/or washing channel(s) such as a luer connection or a hose barb. An insufflation bulb (201) can be connected to the insufflation connection point. Alternatively, other insufflation sources (such as pumps, plumbed pressurized gas, etc) may be connected to the insufflation connection point. This allows for greater flexibility in choice of insufflation apparatus and also allows for replacement of the insufflation apparatus without necessitating replacement of the entire introducer.

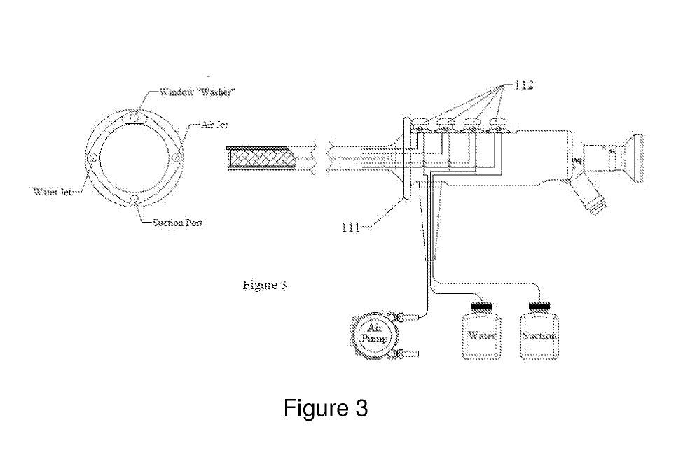

In another embodiment, the proximal end of the introducer may contain a number of valves (112) for controlling the insufflation, aspiration, and wash functions of the introducer. One possible arrangement of separate channels and valves for this purpose, but in no way intended to be limiting, is shown in FIG. 3. Insufflation air and wash water may be supplied by an air pump and water bottle built into the endoscopic illuminator or as standalone components. Alternatively plumbed-in air or CO2, water and vacuum lines in the operating room may be used. Aspiration may be provided by a vacuum pump or similar vacuum source.

The proximal end of the introducer may also have a flange or tabs or handle (111) that facilitate easier handling of the laparoscope and introducer assembly. This handle may also contain a number of valves for controlling the insufflation, suction and wash functions of the introducer. The valves may be deployed in any arrangement that allows for separate and reliable control of the insufflation, wash and aspiration functions. The handle may be positioned at an angle to the main structure of the introducer so that it can be manipulated and operated in a gun fashion.

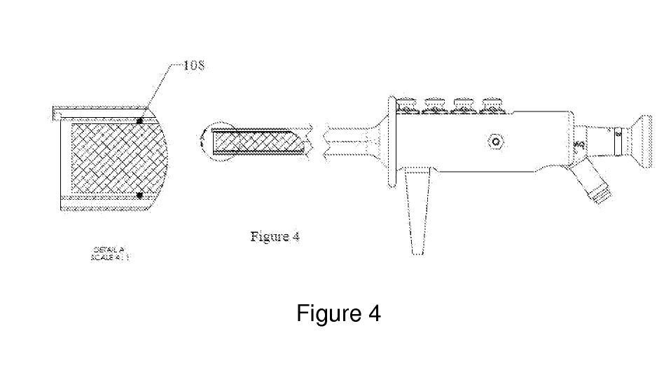

An alternative embodiment may have all of the features of the aforementioned embodiments, except that the main channel at the distal end of the introducer does not have a window that seals and separates the endoscope from the endoluminal space (FIG. 4). In this embodiment, the laparoscope tip is exposed to the endoluminal surface and the introducer contains a circumferential seal (108) between the exterior of the laparoscope and the interior surface of the main channel of the introducer so as to contain the insufflation air within the endoluminal cavity. The seal may be located anywhere along the length of the introducer main channel containing the laparoscope shaft. The seal may be composed of rubber, silicon or other compliant and sufficiently impermeable material. The seal may be in the form of a valve, a wiping seal, 2-stage seal (e.g. a cross slit valve and backup seal) or compliant compression seal (e.g. an O-ring). Such an embodiment may also integrate the insufflation and main channels of the introducer into a single channel. Such an embodiment may also integrate one or more separate ancillary channels to direct a spray of wash water across the tip of the laparoscope or ancillary channels for the irrigation and aspiration of fluids.

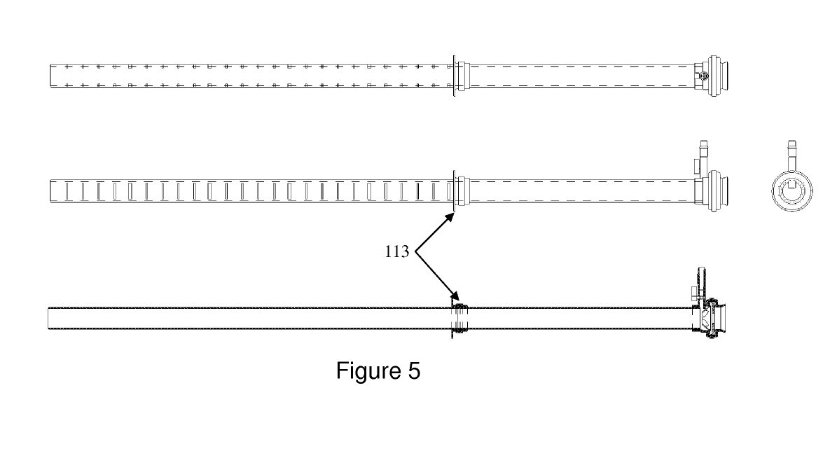

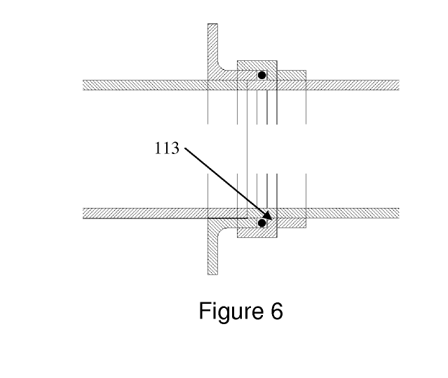

In another embodiment, shown in FIG. 5, the introducer may feature a connection mechanism (113) midway between the distal and proximal ends such that the device may be assembled prior to insertion of the laparoscope. The connection may be in the form of a threaded connection, snap-fit, twist & lock or compression connection and shall prevent leaking of insufflation gas and the transfer of any fluids in ancillary channels. The connection may feature a seal of any type described herein (FIG. 6, 114) so as to maintain insufflation pressure in the connection.

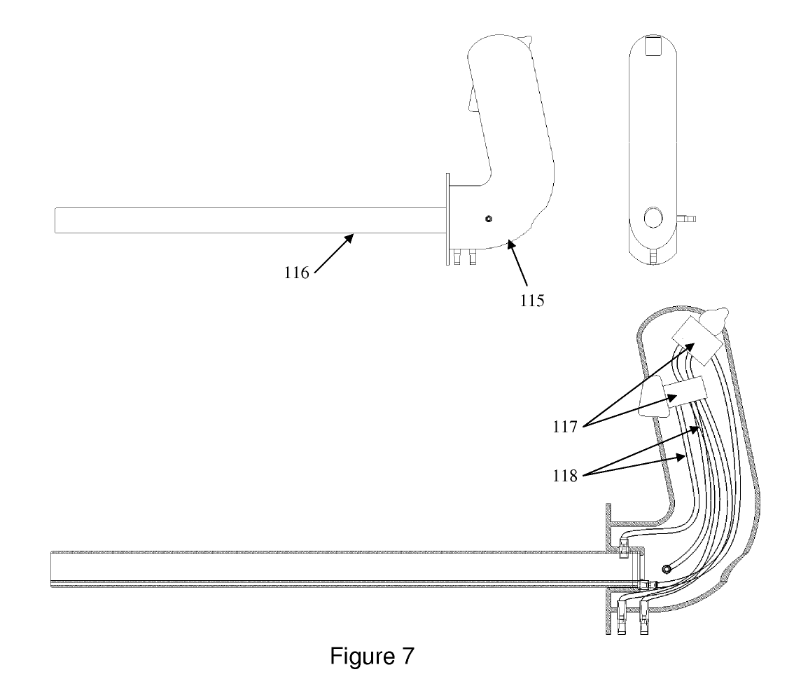

In another embodiment the introducer may feature a removable handle extending at any non-parallel angle in relation to the tube axis. This handle may also contain the valves used to control insufflation, wash/irrigation and aspiration functions. Such an embodiment is shown in FIG. 7. In this embodiment the handle (115) may be a reusable component that attaches to a single-use tube (116) that would be inserted inside the patient. The reusable handle may contain reusable or single-use valves (117) and fluid channels (118) that connect to the main and/or ancillary lumens. The handle may be positioned at an angle to the main structure of the introducer so that it can be manipulated and operated in a gun fashion.

While the invention has been described in the context of examination of an anastomosis or surgical margin in the rectum of a patient following LAR surgery, it will be readily apparent to those of skill in the art that the introducer of the present invention could be used in other contexts. For example, alternate embodiments of the introducer could be deployed in other proximal regions of the bowel or in other body orifices where it would be advantageous to have an introducer that provides multiple channels for imaging and other functionalities (such as irrigation and aspiration) and that provides protection for the surrounding tissue from the surfaces of the laparoscope. As has been described herein in the context of LAR surgery, the alternate embodiments of the introducer could be used in conjunction with a conventional, white-light laparoscope or with an endoscope capable of near infra-red fluorescence illumination and imaging.

While the endoluminal introducer has been illustrated and described in connection with preferred embodiments shown and described in detail, it is not intended to be limited to the details shown since various modifications and structural changes may be made without departing in any way from the scope of the present invention. The embodiments chosen and described explain the principles of the invention and its practical application and do thereby enable a person of skill in the art to best utilize the invention and its various embodiments

What is claimed is:

1. An introducer for use during endoscopic procedures, comprising:
a tube with a proximal end and a distal end and with at least one channel of sufficient diameter to accommodate the passage of an endoscope from the proximal end to the distal end;
an insufflation arrangement configured to pass insufflation gas through a channel in the tube to its distal end; and
an insufflation chamber arrangement configured to maintain insufflation and substantially prevent the insufflation gas from exiting the proximal end of the tube.

2. The introducer of claim 1, wherein the tube is dimensioned to be at least as long as the endoscope so that the endoscope does not extend beyond the distal end of the tube.

3. The introducer of claim 1, wherein the insufflation chamber arrangement comprises a transparent window at the end of the endoscope channel of the introducer.

4. The introducer of claim 1, wherein the insufflation chamber arrangement is a seal between the endoscope and the channel into which the endoscope is inserted.

5. The introducer of claim 1, wherein the insufflation arrangement is an insufflation bulb.

6. The introducer of claim 5, wherein the insufflation bulb is operatively connected to the tube by way of a luer or barbed connection.

7. The introducer of claim 1, wherein the tube comprises a plurality of channels for separate passage of two or more of the endoscope, the insufflation gas, irrigation fluid and aspirated fluid.

8. The introducer of claim 7, further comprising at least one pumping arrangement configured to pass irrigation fluid or aspirating fluid, the pumping arrangement being selected from the group consisting of an air pump, a water pump, a vacuum pump, or a combination thereof.

9. The introducer of claim 1, further comprising a transparent window on the distal end of the tube that is transparent to UV, visible, or infra-red light, but does not allow the endoscope to pass beyond the distal end of the tube.

10. A system for assessing a surgical margin or anastomosis in a patient, comprising:
a tube with a proximal end and a distal end and with at least one channel of sufficient diameter to accommodate the passage of an endoscope from the proximal end to the distal end;
an insufflation arrangement configured to pass insufflation gas through a channel in the tube to its distal end;
an insufflation chamber arrangement configured to maintain insufflation and substantially prevent the insufflation gas from exiting the proximal end of the tube; and
an endoscope deployable into the tube.

11. The system of claim 10, wherein the tube is dimensioned to be at least as long as the endoscope so that the endoscope does not extend beyond the distal end of the tube.

12. The system of claim 10, wherein the insufflation chamber arrangement comprises a transparent window at the end of the endoscope channel of the introducer.

13. The system of claim 10, wherein the insufflation chamber arrangement is a seal between the endoscope and the channel into which it is inserted.

14. The system of claim 10, wherein the insufflation arrangement is an insufflation bulb.

15. The system of claim 14, wherein the insufflation bulb is operatively connected to the tube by way of a luer or barbed connection.

16. The system of claim 10, wherein the tube comprises a plurality of channels for separate passage of two or more of the endoscope, the insufflation gas, irrigation fluid, and aspirated fluid.

17. The system of claim 16, wherein at least one pumping arrangement configured to pass irrigation fluid or aspirating fluid, the pumping arrangement being selected from the group consisting of an air pump, a water pump, a vacuum pump, or a combination thereof.

18. The system of claim 10, further comprising a transparent window on the distal end of the tube that is transparent to UV, visible, or infra-red light, but does not allow the endoscope to pass beyond the distal end of the tube.

19. The system of claim 10, wherein the endoscope is configured to illuminate and image with white light.

20. The system of claim 10, wherein the endoscope is configured to illuminate and image with near infra-red light.

21. The system of claim 10, wherein the endoscope is configured to illuminate and image with white light or near infra-red light or both.
